(19) United States
(12) Patent Application Publication
(10) Pub. No.: US 2018/0253838 A1
(43) Pub. Date: Sep. 6, 2018

(54) SYSTEMS AND METHODS FOR MEDICAL IMAGING OF PATIENTS WITH MEDICAL IMPLANTS FOR USE IN REVISION SURGERY PLANNING

(71) Applicant: MAYO FOUNDATION FOR MEDICAL EDUCATION AND RESEARCH, ROCHESTER, MN (US)

(72) Inventors: JOHN W SPERLING, ROCHESTER, MN (US); MICHAEL LARSON, ROCHESTER, MN (US); BRUCE KLINE, ROCHESTER, MN (US)

(21) Appl. No.: 15/757,127

(22) PCT Filed: Sep. 6, 2016
(86) PCT No.: PCT/US2016/050357

§ 371 Date: Mar. 2, 2018

Related U.S. Application Data
(60) Provisional application No. 62/214,399, filed on Sep. 4, 2015.
(60) Provisional application No. 62/310,305, filed on Mar. 18, 2016.

(51) Int. Cl. G06T 7/00; A61B 6/00; A61B 5/055; A61B 5/00; A61B 5/107; A61B 8/08; G06T 7/70; G06T 17/00; G06T 11/00; A61B 34/10; A61F 2/30

(52) U.S. Cl. CPC G06T 7/0012; A61F 2002/30948; A61B 5/055; A61B 6/5258; A61B 5/4509; A61B 5/1073; A61B 6/5247; A61B 8/5261; G06T 7/70; G06T 17/00; G06T 11/008; A61B 34/10; A61F 2/30942; G06T 2207/10081; G06T 2207/10088; G06T 2207/20056; G06T 2207/20221; G06T 2207/30008; G06T 2207/30052; G06T 2207/10132; A61B 2034/108; A61B 6/5252

(57) ABSTRACT

Systems and methods are provided for processing medical images to generate information useful for planning or guiding revision surgeries, designing implants for use in revisions surgeries, or generally evaluating the bone architecture of a subject. The medical images may be x-ray images, such as those acquired with a computed tomography (“CT”) system, magnetic resonance images, such as those acquired with a magnetic resonance imaging (“MRI”) system, or ultrasound images, such as those acquired with an ultrasound imaging system. The images can also be fused together, or otherwise combined, to produce combined images that enhance the depiction of an instrument or implant in the subject relative to the uncombined images.

### CROSS-REFERENCE TO RELATED APPLICATIONS

This application claims the benefit of U.S. Provisional Patent Application 62/214,399, filed Sep. 4, 2015, entitled “Systems and Methods for Improved Imaging and Treatment of Patients with Medical Implants” and U.S. Provisional Patent Application 62/310,305, filed Mar. 18, 2016, entitled “Systems and Methods for Medical Imaging of Patients with Medical Implants for Use in Revision Surgery Planning.” All of which are incorporated herein by reference for all purposes.

### BACKGROUND OF THE DISCLOSURE

The field of the invention relates to medical imaging, and more particularly to medical imaging, such as x-ray imaging or magnetic resonance imaging (“MRI”), for use in planning revision surgeries or designing implants to be used therein.

In the United States alone, there were 719,000 total knee arthroplasties and 332,000 hip replacements performed in 2014. By 2030, the demand for primary total hip arthroplasties is estimated to grow to 572,000 and the demand for primary total knee arthroplasties is projected to grow to 3.48 million procedures. The demand for hip and knee revision procedures is also projected to increase dramatically due to primary procedures being performed on younger patients, and due to an increase in obesity leading to faster wear with subsequent failure. Additional areas of orthopedic surgery are seeing significant increases in volume. The rate of shoulder arthroplasty is growing at five times that of knee and hip arthroplasty, with over 100,000 procedures performed annually in the United States. There has also been a dramatic increase in the rate of spinal surgery with instrumentation, as well as revision spinal surgery. As increasing healthcare resources become available in developing countries, there is significant growth in the burden of revision surgery throughout the world.

Presently, the ability to accurately plan revision surgeries is lacking. For instance, one of the central challenges facing the surgeon in orthopedic revision procedures is quantifying the amount of remaining bone stock and specific bone architecture. This information is critical in determining whether to proceed with surgery, as well as planning for appropriate components. However, the surgeon is frequently left unsure if there is enough bone remaining to perform a revision, and has limited ability to plan for proper components. This is because planning revisions often suffer from limited diagnostic images. In fact, at some institutions without dedicated musculoskeletal radiologists, image quality can be so poor that the images have essentially no diagnostic value, forcing the clinician to utilize a best guess analysis.

Metallic, plastic, and other implanted materials commonly present in subjects receiving CT examinations for revision surgeries, and can produce severe image artifacts in the form of streaks, shadows, and distortions, thus preventing accurate identification of underlying anatomy. Image artifacts generally arise from the data inconsistency between ideal models assumed by reconstruction algorithms and the actual CT signal, which has been contaminated by the metal, or other highly attenuating material. X-rays are highly attenuated by metals and other materials, which in turn amplifies factors that lead to data inconsistencies and, eventually, to image artifacts such as noise, beam hardening, scattering, and nonlinear partial volume effects. In this manner, small implanted objects that may occupy only a small image region can produce artifacts that affect entire images, obscuring anatomical structures.

In addition, personalized devices have become increasingly popular for primary arthroplasty, and other procedures. Such patient specific instrumentation can help improve the ability to place components in the correct alignment, as well as plan for the precise components to be used at surgery. However, current imaging techniques are significantly limited in the ability to make patient specific guides for revision cases. There have been scattered case reports on CT scans being used for a custom guide in the revision setting; however, there are currently no large orthopedic manufacturers that offer patient specific instrumentation for revision surgery. The current CT scans have too much artifact to accurately plan such guides. In cases where a surgeon would consider making a patient specific guide, significant assumptions must be made and the process is extremely labor intensive.

Despite efforts, image artifacts continue to pose severe problems in the clinic for various diagnostic and interventional procedures, and particularly for revision surgery applications. Therefore, there remains a need for improved systems and methods for imaging a patient with prior implants or instrumentation.

### SUMMARY OF THE DISCLOSURE

The present disclosure overcomes the aforementioned drawbacks by providing systems and methods for improved imaging of patients with implants and instrumentation present within a patient's anatomy. In some aspects, implanted objects or instrumentation are identified and removed from imaging data, allowing for clear identification of underlying tissue, such as remaining bone tissue. This would facilitate more accurate clinical diagnosis, as well as improved design and manufacturing of patient specific implants and guides for revision surgeries.

It is one aspect of the present invention to provide a method for generating a report that provides information about a revision surgery plan or guide based on image data acquired with a medical imaging system, which may be an x-ray imaging system, a magnetic resonance imaging (“MRI”) system, or the like. Image data of a subject acquired with a medical imaging system is provided to a computer system. The provided image data is processed with the computer system to identify at least one object implanted in the subject's anatomy and to remove the identified at least one object from the image data. A report is then generated with the computer system. The report is based on the processed image data and provides information about a revision surgery plan specific to the subject.

It is another aspect of the present invention to provide a method for generating a report that provides information for designing an implant for use in a revision surgery based on image data acquired with a medical imaging system, which may be an x-ray imaging system, an MRI system, or the like. Image data of a subject acquired with a medical imaging system is provided to a computer system. The provided image data is processed with the computer system to identify at least one object implanted in the subject's anatomy and to remove the identified at least one object from the image data. A report is then generated with the computer system. The report is based on the processed image data and provides information for designing a subject-specific implant for use in a revision surgery.

It is still another aspect of the present invention to provide a method for generating a report that provides information about a subjects bone architecture based on image data acquired with a medical imaging system, which may be an x-ray imaging system, an MRI system, or the like. Image data of a subject acquired with a medical imaging system is provided to a computer system. The provided image data is processed with the computer system to identify at least one object implanted in the subject's anatomy and to remove the identified at least one object from the image data. A report is then generated with the computer system. The report is based on the processed image data and provides information about the subject's bone architecture.

It is still another aspect of the present invention to provide a method for generating a report that provides information about a revision surgery plan, revision surgery guide, subject-specific implant for use in a revision surgery, or the subject's bone architecture based on image data acquired with one or more medical imaging systems. The method includes providing, to a computer system, image data of a subject acquired with at least one medical imaging system. Image fusion data is generated by combining the image data with the computer system, whereby the image fusion data enhances a depiction of at least one object implanted in the subject's anatomy relative to the image data. A report is then generated with the computer system based on the image fusion data. This report provides information about at least one of a revision surgery plan specific to the subject, designing a subject-specific implant for use in a revision surgery, or the subject's bone architecture.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is an illustration of an example CT imaging system.

FIG. 1B is a block diagram of an example CT imaging system.

FIG. 2 is a block diagram of an example of a magnetic resonance imaging (“MRI”) system.

FIG. 3 is a flowchart setting forth the steps of an example method for generating a report based on medical imaging data, for use in revision surgery planning or implant design.

FIG. 4 is a flowchart setting forth the steps of an example method for generating a report based on medical imaging data that is fused together, or otherwise combined, for use in revision surgery planning or implant design.

FIG. 5 is an example of a computer system that can implement the methods described herein.

### DETAILED DESCRIPTION

Described here are systems and methods for processing medical images to generate information useful for planning or guiding revision surgeries, designing implants for use in revision surgeries, or generally evaluating the bone architecture of a subject. In some aspects, the medical images can be x-ray images, such as those acquired with a computed tomography (“CT”) system, a C-arm x-ray imaging system, a plane x-ray imaging system, and so on. In some other aspects, the medical images can be magnetic resonance images acquired with a magnetic resonance imaging (“MRI”) system. In still other aspects, the medical images can be ultrasound images acquired with an ultrasound system.

Revision surgery in the setting of prior instrumentation or implants are among the most challenging and expensive surgical cases. Unlike the primary setting where cases can be accurately planned, the surgeon frequently enters a revision case with minimal information. In some instances, the surgeon is left to guess what type of components may be needed for a particular revision surgery, or may be left to question whether there will be enough bone to place new components. Currently, there is an unaddressed market need to understand the underlying bone architecture among these patients.

Approximately twenty percent of the subjects scanned with CT every year in the United States contain metal implants. Accordingly, there is a rapidly growing clinical need for improved imaging of patients with prior implants and instrumentation, especially in relation to the performance of revision surgeries. The systems and methods described here can be used in a widespread manner on various clinical CT scanners to help surgeons and other clinicians evaluate remaining bone stock in patients undergoing revision surgery. Applications of the systems and methods described here may thus span various areas of orthopedic surgery, neurosurgery, otolaryngology, dentistry, and so forth.

The system and methods described here can facilitate creating improved protocols for imaging patients with implants or instrumentation. Various implementations include imaging systems and software solutions for removing implants or instrumentation—and associated image artifacts—from x-ray images, which in turn allows for more accurate visualization of underlying bone and other tissues. For instance, the systems and methods described here can be designed to perform automated metal, ceramic, or plastic implant (with or without cement) subtraction and segmentation. With the cost of performing revision arthroplasty ranging between $50,000 to greater than $100,000, having information about bone architecture prior to surgery would be extremely valuable.

The systems and methods described here also provide for patient specific guides for revision surgery. The rate of failure of revision surgery is often higher than primary surgery. Due to severe scarring and distorted anatomy from previous surgeries, determining proper alignment can be very difficult in revision surgeries. Personalized guides, based on accurate depiction of underlying anatomical structures (e.g., bone architecture) would help dramatically in improving the rates of success for revision surgery. In fact, studies have shown that the highest benefits of patient specific guides is often in cases with bone loss and distorted anatomy due primary medical procedures.

3D printing has evolved to be a powerful technology in planning primary surgical procedures by allowing the production of patient-specific anatomical models, instrumentation to place components in accurate positions, and custom, patient-specific implants. However, in patients with instrumentation or implants in place, metal artifacts do not allow for complete image segmentation and, therefore, interpolation is often necessary. This interpolation, however, results in guessing where the true underlying bone may be located, which leads to greater potential for error in model design, instrument design, and implant design.

Thus, in addition to the applications mentioned above, the systems and methods described here also provide for improved accuracy of design and manufacturing of implants for revision surgery. In particular, improved pre-operative imaging would facilitate revision surgeries by allowing determination of which implants may be needed at the revision, or if custom implants would need to be manufactured. In the latter case, personalized implants with improved design could be achieved due to the improved image quality. On a larger scale, higher quality images could be used to focus development of implants used for revision, including specific sizes and shapes would be needed.

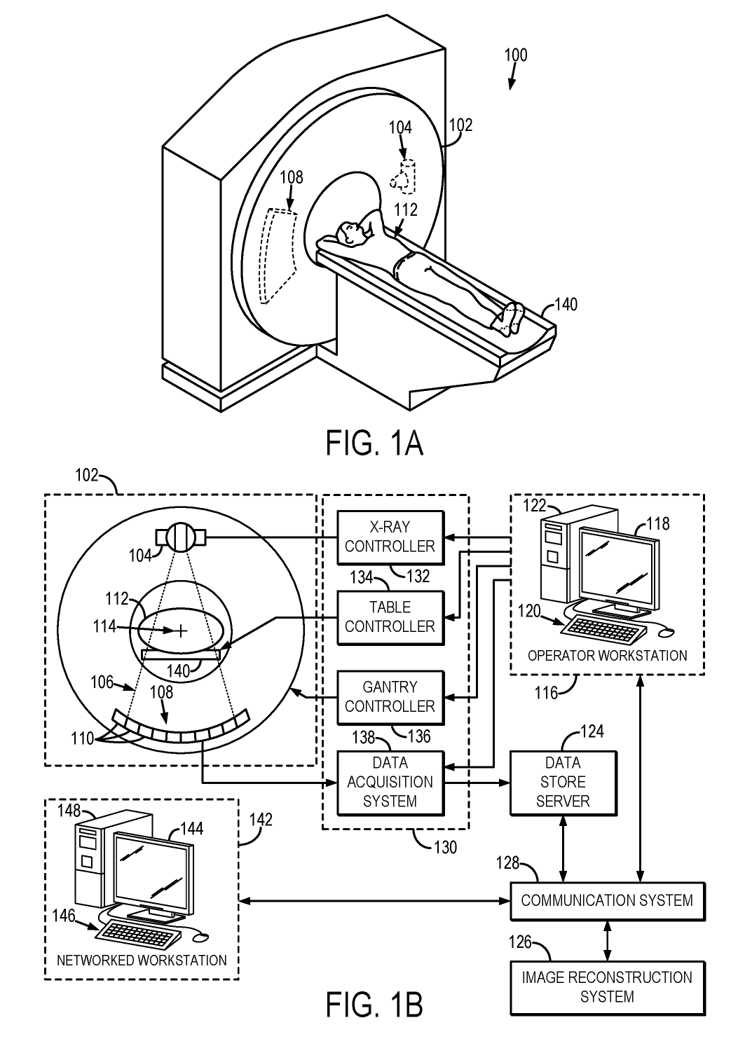

Referring particularly now to FIGS. 1A and 1B, an example of an x-ray computed tomography (“CT”) imaging system 100 is illustrated. The CT system includes a gantry 102, to which at least one x-ray source 104 is coupled. The x-ray source 104 projects an x-ray beam 106, which may be a fan-beam or cone-beam of x-rays, towards a detector array 108 on the opposite side of the gantry 102. The detector array 108 includes a number of x-ray detector elements 110. Together, the x-ray detector elements 110 sense the projected x-rays 106 that pass through a subject 112, such as a medical patient or an object undergoing examination, that is positioned in the CT system 100. Each x-ray detector element 110 produces an electrical signal that may represent the intensity of an impinging x-ray beam and, hence, the attenuation of the beam as it passes through the subject 112. In some configurations, each x-ray detector 110 is capable of counting the number of x-ray photons that impinge upon the detector 110. During a scan to acquire x-ray projection data, the gantry 102 and the components mounted thereon rotate about a center of rotation 114 located within the CT system 100.

The CT system 100 also includes an operator workstation 116, which typically includes a display 118; one or more input devices 120, such as a keyboard and mouse; and a computer processor 122. The computer processor 122 may include a commercially available programmable machine running a commercially available operating system. The operator workstation 116 provides the operator interface that enables scanning control parameters to be entered into the CT system 100. In general, the operator workstation 116 is in communication with a data store server 124 and an image reconstruction system 126. By way of example, the operator workstation 116, data store server 124, and image reconstruction system 126 may be connected via a communication system 128, which may include any suitable network connection, whether wired, wireless, or a combination of both. As an example, the communication system 128 may include both proprietary or dedicated networks, as well as open networks, such as the internet.

The operator workstation 116 is also in communication with a control system 130 that controls operation of the CT system 100. The control system 130 generally includes an x-ray controller 132, a table controller 134, a gantry controller 136, and a data acquisition system 138. The x-ray controller 132 provides power and timing signals to the x-ray source 104 and the gantry controller 136 controls the rotational speed and position of the gantry 102. The table controller 134 controls a table 140 to position the subject 112 in the gantry 102 of the CT system 100.

The DAS 138 samples data from the detector elements 110 and converts the data to digital signals for subsequent processing. For instance, digitized x-ray data is communicated from the DAS 138 to the data store server 124. The image reconstruction system 126 then retrieves the x-ray data from the data store server 124 and reconstructs an image therefrom. The image reconstruction system 126 may include a commercially available computer processor, or may be a highly parallel computer architecture, such as a system that includes multiple-core processors and massively parallel, high-density computing devices. Optionally, image reconstruction can also be performed on the processor 122 in the operator workstation 116. Reconstructed images can then be communicated back to the data store server 124 for storage or to the operator workstation 116 to be displayed to the operator or clinician.

The CT system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 116, or in a different facility, such as a different healthcare institution or clinic.

The networked workstation 142, whether within the same facility or in a different facility as the operator workstation 116, may gain remote access to the data store server 124 and/or the image reconstruction system 126 via the communication system 128. Accordingly, multiple networked workstations 142 may have access to the data store server 124 and/or image reconstruction system 126. In this manner, x-ray data, reconstructed images, or other data may be exchanged between the data store server 124, the image reconstruction system 126, and the networked workstations 142, such that the data or images may be remotely processed by a networked workstation 142. This data may be exchanged in any suitable format, such as in accordance with the transmission control protocol (“TCP”), the internet protocol (“IP”), or other known or suitable protocols.

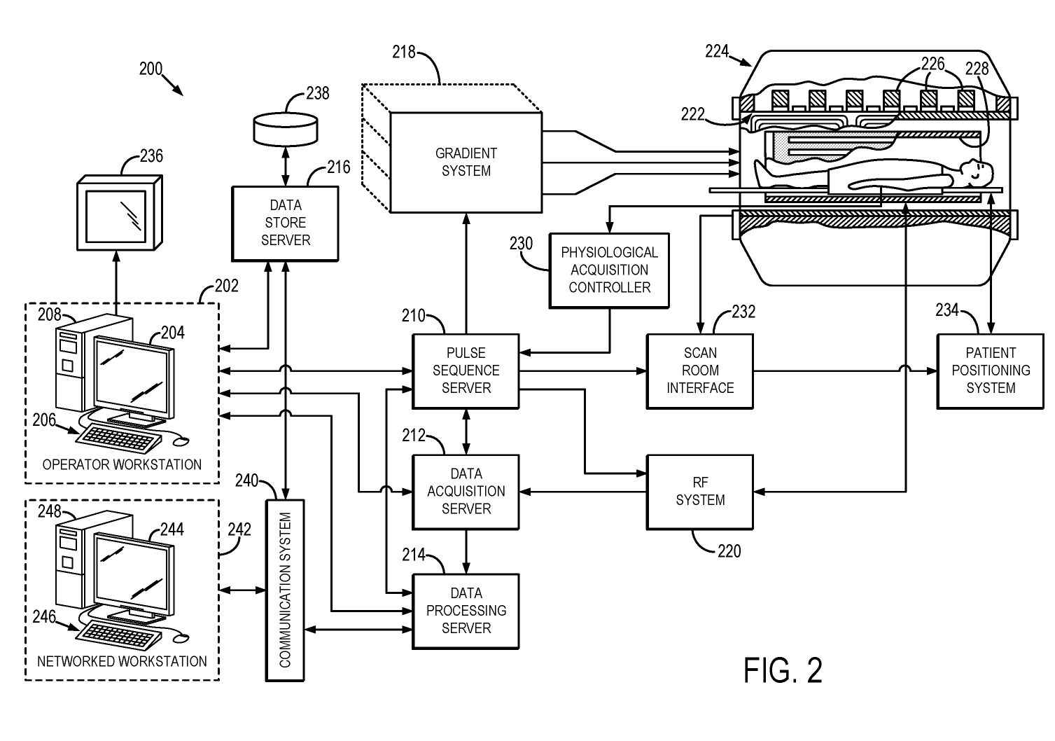

Referring particularly now to FIG. 2, an example of a magnetic resonance imaging (“MRI”) system 200 is illustrated. The MRI system 200 includes an operator workstation 202, which will typically include a display 204; one or more input devices 206, such as a keyboard and mouse; and a processor 208. The processor 208 may include a commercially available programmable machine running a commercially available operating system. The operator workstation 202 provides the operator interface that enables scan prescriptions to be entered into the MRI system 200. In general, the operator workstation 202 may be coupled to four servers: a pulse sequence server 210; a data acquisition server 212; a data processing server 214; and a data store server 216. The operator workstation 202 and each server 210, 212, 214, and 216 are connected to communicate with each other. For example, the servers 210, 212, 214, and 216 may be connected via a communication system 240, which may include any suitable network connection, whether wired, wireless, or a combination of both. As an example, the communication system 240 may include both proprietary or dedicated networks, as well as open networks, such as the internet.

The pulse sequence server 210 functions in response to instructions downloaded from the operator workstation 202 to operate a gradient system 218 and a radiofrequency (“RF”) system 220. Gradient waveforms necessary to perform the prescribed scan are produced and applied to the gradient system 218, which excites gradient coils in an assembly 222 to produce the magnetic field gradients Gₓ, G_{y}, and G_{z} used for position encoding magnetic resonance signals. The gradient coil assembly 222 forms part of a magnet assembly 224 that includes a polarizing magnet 226 and a whole-body RF coil 228.

RF waveforms are applied by the RF system 220 to the RF coil 228, or a separate local coil (not shown in FIG. 2), in order to perform the prescribed magnetic resonance pulse sequence. Responsive magnetic resonance signals detected by the RF coil 228, or a separate local coil (not shown in FIG. 2), are received by the RF system 220, where they are amplified, demodulated, filtered, and digitized under direction of commands produced by the pulse sequence server 210. The RF system 220 includes an RF transmitter for producing a wide variety of RF pulses used in MRI pulse sequences. The RF transmitter is responsive to the scan prescription and direction from the pulse sequence server 210 to produce RF pulses of the desired frequency, phase, and pulse amplitude waveform. The generated RF pulses may be applied to the whole-body RF coil 228 or to one or more local coils or coil arrays (not shown in FIG. 2).

The RF system 220 also includes one or more RF receiver channels. Each RF receiver channel includes an RF preamplifier that amplifies the magnetic resonance signal received by the coil 228 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (1)

and the phase of the received magnetic resonance signal may also be determined according to the following relationship:

$$ϕ = {tan}^{- 1} ( \frac{Q}{I} ) . \qquad ( 2 )$$

The pulse sequence server 210 also optionally receives patient data from a physiological acquisition controller 230. By way of example, the physiological acquisition controller 230 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 210 to synchronize, or “gate,” the performance of the scan with the subject's heart beat or respiration.

The pulse sequence server 210 also connects to a scan room interface circuit 232 that receives signals from various sensors associated with the condition of the patient and the magnet system. It is also through the scan room interface circuit 232 that a patient positioning system 234 receives commands to move the patient to desired positions during the scan.

The digitized magnetic resonance signal samples produced by the RF system 220 are received by the data acquisition server 212. The data acquisition server 212 operates in response to instructions downloaded from the operator workstation 202 to receive the real-time magnetic resonance data and provide buffer storage, such that no data is lost by data overrun. In some scans, the data acquisition server 212 does little more than pass the acquired magnetic resonance data to the data processor server 214. However, in scans that require information derived from acquired magnetic resonance data to control the further performance of the scan, the data acquisition server 212 is programmed to produce such information and convey it to the pulse sequence server 210. For example, during prescans, magnetic resonance data is acquired and used to calibrate the pulse sequence performed by the pulse sequence server 210. As another example, navigator signals may be acquired and used to adjust the operating parameters of the RF system 220 or the gradient system 218, or to control the view order in which k-space is sampled. In still another example, the data acquisition server 212 may also be employed to process magnetic resonance signals used to detect the arrival of a contrast agent in a magnetic resonance angiography (“MRA”) scan. By way of example, the data acquisition server 212 acquires magnetic resonance data and processes it in real-time to produce information that is used to control the scan.

The data processing server 214 receives magnetic resonance data from the data acquisition server 212 and processes it in accordance with instructions downloaded from the operator workstation 202. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 214 are conveyed back to the operator workstation 202 where they are stored. Real-time images are stored in a data base memory cache (not shown in FIG. 2), from which they may be output to operator display 212 or a display 236 that is located near the magnet assembly 224 for use by attending physicians. Batch mode images or selected real time images are stored in a host database on disc storage 238. When such images have been reconstructed and transferred to storage, the data processing server 214 notifies the data store server 216 on the operator workstation 202. The operator workstation 202 may be used by an operator to archive the images, produce films, or send the images via a network to other facilities.

The MRI system 200 may also include one or more networked workstations 242. By way of example, a networked workstation 242 may include a display 244; one or more input devices 246, such as a keyboard and mouse; and a processor 248. The networked workstation 242 may be located within the same facility as the operator workstation 202, or in a different facility, such as a different healthcare institution or clinic.

The networked workstation 242, whether within the same facility or in a different facility as the operator workstation 202, may gain remote access to the data processing server 214 or data store server 216 via the communication system 240. Accordingly, multiple networked workstations 242 may have access to the data processing server 214 and the data store server 216. In this manner, magnetic resonance data, reconstructed images, or other data may be exchanged between the data processing server 214 or the data store server 216 and the networked workstations 242, such that the data or images may be remotely processed by a networked workstation 242. This data may be exchanged in any suitable format, such as in accordance with the transmission control protocol (“TCP”), the internet protocol (“IP”), or other known or suitable protocols.

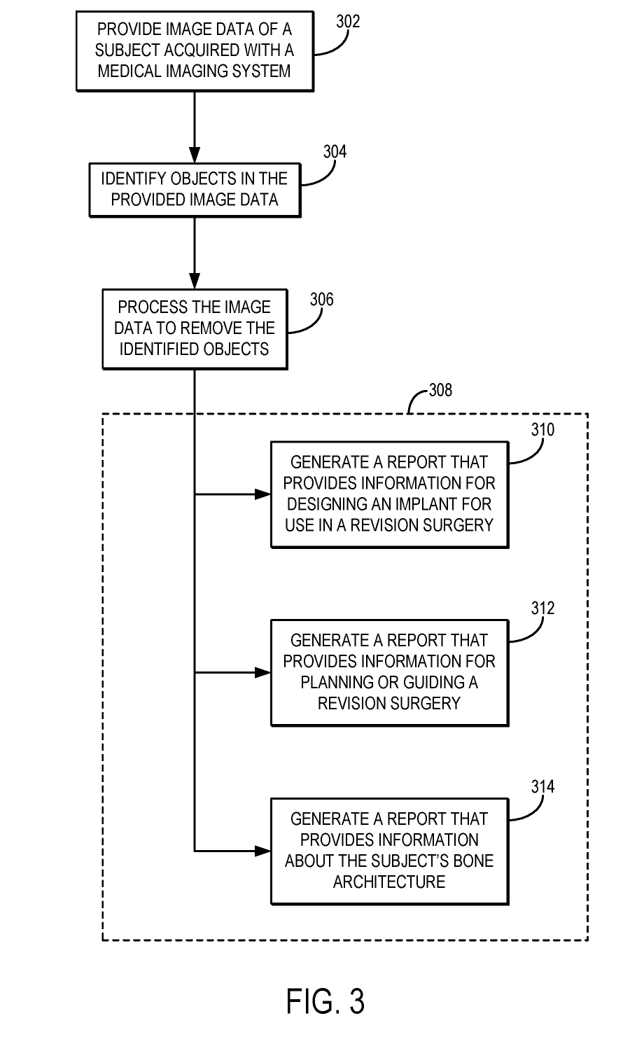

Referring now to FIG. 3, a flowchart is illustrated as setting forth the steps of an example method for generating a report based on medical image data, wherein the report provides information useful for resection surgeries. The method includes providing image data, which may include reconstructed images or associated data, acquired with a medical imaging system to a computer processor, as indicated at step 302. The medical imaging system can be an x-ray imaging system or an MRI system. As one example, the x-ray imaging system can be a CT imaging system, such as the one illustrated in FIGS. 1A and 1B. In some instances, the CT imaging system can be a dual-energy CT imaging system, in which case the provided images can be representative of two different x-ray energies. In other examples, the x-ray imaging system can be a C-arm x-ray imaging system, a digital radiography system, or so on. Associated data may be, for instance, x-ray attenuation data acquired by an x-ray imaging system, or raw k-space data acquired with an MRI system.

In some aspects, providing image data includes retrieving previously acquired images or data from a memory or other data storage device. In other aspects, however, providing the image data includes acquiring the images or data with the medical imaging system.

Preferably, the image data are acquired using acquisition techniques that minimize image artifacts, or the acquired image data are processed to minimize image artifacts or otherwise improve image quality. In some instances, the acquired image data are processed to remove artifacts or to improve signal-to-noise ratio (“SNR”) before or during image reconstruction. In some other instances, the already reconstructed images are processed to remove artifacts or to improve SNR.

As one example, the data acquired using various projection views may be denoised using a locally adaptive bilateral filter prior to image reconstruction, as described in U.S. Pat. No. 8,965,078, which is herein incorporated by reference in its entirety. As another example, images may be denoised using a modified non-local means (“NLM”) algorithm that is adaptive to local variations of noise levels, as described in U.S. Pat. No. 9,036,771, which is herein incorporated by reference in its entirety.

As one example for when and MRI system is used to acquire the image data, a data acquisition that minimizes artifacts attributable to metallic implants can be used. For example, pulse sequences that minimize the susceptibility-induced artifacts can be implemented. As another example, imaging techniques, such as multi-acquisition variable-resonance image combination (“MAVRIC”) or slice encoding for metal artifact correction can be used.

Referring again to FIG. 3, objects are identified in the provided image data, as indicated at step 304. Such objects can include metallic implants, plastic implants, or other implants or instrumentation composed of materials that significantly attenuate x-rays or confound magnetic resonance images. In some applications, identifying such objects includes identifying regions-of-interest (“ROIs”) in the image data that contain the objects. In some aspects, various material decomposition techniques may be applied to identify the objects or implants based on data prior to reconstruction, or based on reconstructed images. In some other applications, identifying such objects can include implementing image segmentation algorithms.

As one example, a method for determining the distribution of density and constituent material concentration throughout an imaged object can be used to identify objects in the image. Such a method is described, for instance, in U.S. Pat. No. 7,885,373, which is herein incorporated by reference in its entirety. This approach generally includes converting dual-energy image data to attenuation coefficients associated with each of the energy levels, calculating a ratio of the attenuation coefficients with one energy level to the attenuation coefficients associated with another energy level, and correlating the calculated ratio to indicate a concentration of a constituent material in the imaged object. Material decomposition of more than two constituent materials may also be performed, as described in U.S. Pat. No. 8,290,232, which is herein incorporated by reference in its entirety. In this latter approach, mass attenuation coefficients associated with each energy level are expressed as a the product determined effective densities and a sum of constituent materials mass attenuation coefficients weighed by respective concentrations of the constituent materials.

In this manner, objects, including metallic or plastic implants or instrumentations can be identified. Using information associated with the identified objects, the provided image data may then be processed, as indicated at step 306, to subtract or otherwise remove the identified objects in order to produce images and other suitable information for diagnostic and treatment purposes. For instance, the identified objects may be removed from reconstructed images using a number of segmentation techniques known in the art.

By way of example, images in which identified objects are removed may be produced using methods described in U.S. Pat. No. 8,280,135, which is herein incorporated by reference in its entirety. In this example technique, reformatted projections are produced using the data acquired at a common projection angle, which are then processed to detect and segment regions corresponding to objects composed of metals, metal alloys, and other highly-attenuating materials, as well as plastics and other materials. Segmented regions associated with metallic implants, for example, can then be removed from the reformatted projections and replaced with interpolated information to produce corrected projections for use in reconstructing images in which the identified objects have been removed.

Based on the image data from which the identified objects have been removed, one or more reports are generated by the computer system, as indicated generally at process block 308. In some aspects, the generated report can provide information for planning or otherwise guiding a revision surgery, as indicated at step 310. For instance, the report can include information, such as images, data, or information derived therefrom, that can be used for planning or otherwise guiding a revision surgery. As an example, the generated report can indicate a patient-specific revision surgery guide, which may indicate an optimal plan for performing revision surgery for a particular subject based on that subject's anatomy, including their bone architecture following previous surgeries, as well as the existing implants or instrumentation present in the subject. As another example, the report can include a computer-generated model of the subject's bone, surrounding anatomy, or both.

In some other aspects, the generated report can provide information for designing an implant for use in a revision surgery, as indicated at step 312. For instance, the report can include information, such as images, data, or information derived therefrom, that can be used to design a patient-specific implant for use in a revision surgery. Such a report can advantageously provide information about the subject's anatomy, including the bone architecture following previous surgeries, which can in turn be used to design a custom implant specifically tailored to the subject's anatomy. Thus, as one example, the report can include a computer-generated model of the subject's bone or a computer-generated model of an implant designed specifically for the subject's anatomy. In some embodiments, the computer-generated model of an implant can include data formatted to be provided to a computer numerical control (“CNC”) system, a three-dimensional printer, or any other suitable system that is configured to machine or otherwise construct a designed implant.

In still other aspects, the generated report can provide information about the subject's bone architecture, generally, as indicated at step 314. For instance, the report can include information, such as images, data, or information derived therefrom, that indicates a bone architecture of the subject, such as a bone density or a bone volume, as well as information about other tissues. Such a report can be advantageous for patients undergoing revision surgery, whereby information about the remaining bone or bone quality can be utilized to accurately plan for and execute a revision surgery. As another example, the report can include a computer-generated model of the subject's bone, surrounding anatomy, or both.

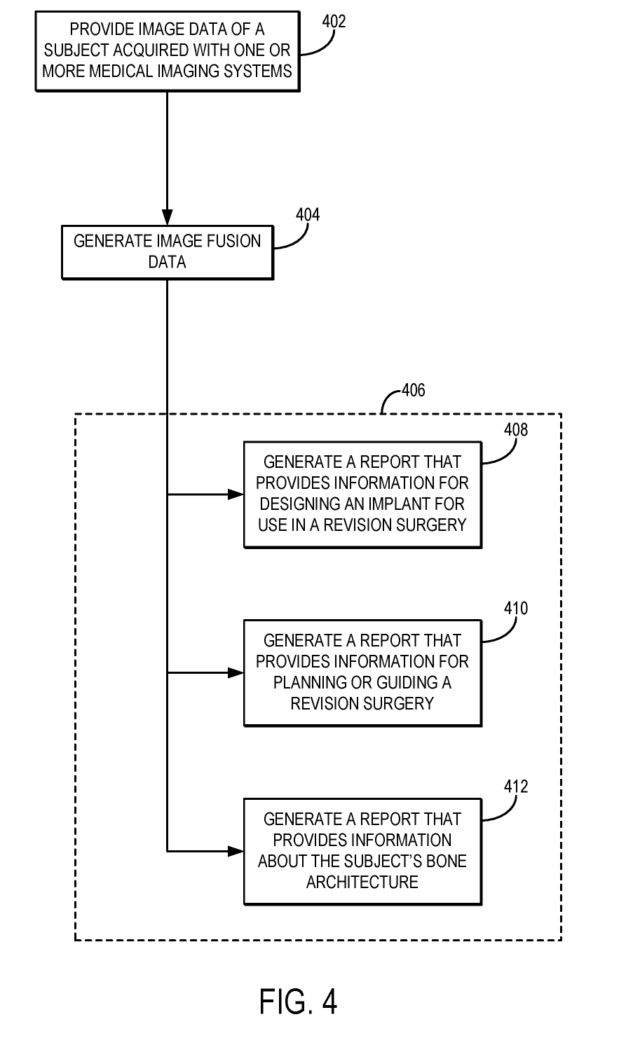

Referring now to FIG. 4, a flowchart is illustrated as setting forth the steps of an example method for generating a report based on medical image data, wherein the report provides information useful for resection surgeries. It is noted, however, that in addition to benefitting revision surgeries, the methods described here can be beneficial for primary patients to improve image quality, such as improved visualization of cortical margins.

The method includes providing image data, which may include reconstructed images or associated data, acquired with one or more medical imaging systems to a computer processor, as indicated at step 402. The one or more medical imaging systems can include an x-ray imaging system, an MRI system, an ultrasound imaging system, and so on. As one example, the x-ray imaging system can be a CT imaging system, such as the one illustrated in FIGS. 1A and 1B. In some instances, the CT imaging system can be a dual-energy CT imaging system, in which case the provided images can be representative of two different x-ray energies. In other examples, the x-ray imaging system can be a C-arm x-ray imaging system, a digital radiography system, or so on. Associated data may be, for instance, x-ray attenuation data acquired by an x-ray imaging system, or raw k-space data acquired with an MRI system.

In some aspects, providing image data includes retrieving previously acquired images or data from a memory or other data storage device. In other aspects, however, providing the image data includes acquiring the images or data with the one or more medical imaging systems.

Preferably, the image data are acquired using acquisition techniques that minimize image artifacts, or the acquired image data are processed to minimize image artifacts or otherwise improve image quality. In some instances, the acquired image data are processed to remove artifacts or to improve signal-to-noise ratio (“SNR”) before or during image reconstruction. In some other instances, the already reconstructed images are processed to remove artifacts or to improve SNR.

The image data from one or more medical imaging systems can be fused together, or otherwise combined, to generate image fusion data in which artifacts in subjects with prior instrumentation or implants are eliminated or otherwise reduced, as indicated at step 404.

In some aspects, different imaging modalities (e.g., CT, MRI, tomosynthesis, plain radiographs, ultrasound), each with artifacts, but dissimilar artifacts, can be fused together or otherwise combined to generate combined image data that can eliminate or significantly decrease artifacts. As one particular example, combined image data can include fusing, or otherwise combining, magnetic resonance images with x-ray CT images. The magnetic resonance images depict soft tissue better than the x-ray CT images, whereas the x-ray CT images depict bone better than the magnetic resonance images. Thus, an image fusion approach may be used to best visualize both the soft tissues and bones in an anatomy of interest.

In some other aspects, the image fusion data is not generated from multiple different imaging modalities, but can be generated by fusing together, or otherwise combining, image data from the same imaging modality, but processed in different ways. As one example, the image fusion data can including fusing together, or otherwise combining, a first image, which may be an x-ray CT image, reconstructed in a conventional fashion and a second image reconstructed using a metal artifact reduction protocol. In this way, the resulting image fusion data may have preserved Hounsfield Units in a region-of-interest, and can also have a generally denoised appearance. Corrections may also be constrained to a narrow region-of-interest in the image (e.g., constrained to where metal artifacts are present), while the remaining image space is processed as normal.

In some instances, the combination of image data can be optimized. As an example, what data to combine, from which modality to combine data, and how that data can be modified to reduce metal artifacts or other artifacts can be optimized using a comparison to a database of ideal image fusion cases, or by metrics of prospective image quality for the resulting combination. Additionally, images or other data acquired from phantoms with instrumentation or implants can be used as a part of the optimization to further refine the different modalities and the specific algorithms.

The image data to be combined can be manipulated during acquisition, reconstruction, pre-processing, post-processing, and so on. As one example, CT data may have been acquired with a specialized protocol designed to reduce metal artifacts, and this image data may be combined with MR data that has been post-processed to reduce artifacts and increase tissue contrast.

Optionally, objects can be identified in the provided image data or the image fusion data. Such objects can include metallic implants, plastic implants, or other implants or instrumentation composed of materials that significantly attenuate x-rays or confound magnetic resonance images. In some applications, identifying such objects includes identifying ROIs in the image data that contain the objects. In some aspects, various material decomposition techniques may be applied to identify the objects or implants based on data prior to reconstruction, or based on reconstructed images. In some other applications, identifying such objects can include implementing image segmentation algorithms.

In this manner, objects, including metallic or plastic implants or instrumentations can be identified. Using information associated with the identified objects, the provided image data, or the image fusion data, may then be processed to subtract or otherwise remove the identified objects in order to produce images and other suitable information for diagnostic and treatment purposes. For instance, the identified objects may be removed from reconstructed images using a number of segmentation techniques known in the art.

Based on the image fusion data, one or more reports are generated by the computer system, as indicated generally at process block 406. In some aspects, the generated report can provide information for planning or otherwise guiding a revision surgery, as indicated at step 408. For instance, the report can include information, such as images, data, or information derived therefrom, that can be used for planning or otherwise guiding a revision surgery. As an example, the generated report can indicate a patient-specific revision surgery guide, which may indicate an optimal plan for performing revision surgery for a particular subject based on that subject's anatomy, including their bone architecture following previous surgeries, as well as the existing implants or instrumentation present in the subject. As another example, the report can include a computer-generated model of the subject's bone, surrounding anatomy, or both.

In some other aspects, the generated report can provide information for designing an implant for use in a revision surgery, as indicated at step 410. For instance, the report can include information, such as images, data, or information derived therefrom, that can be used to design a patient-specific implant for use in a revision surgery. Such a report can advantageously provide information about the subject's anatomy, including the bone architecture following previous surgeries, which can in turn be used to design a custom implant specifically tailored to the subject's anatomy. Similarly, a patient-specific anatomical model can also be designed. Thus, as one example, the report can include a computer-generated model of the subject's bone or a computer-generated model of an implant designed specifically for the subject's anatomy. In some embodiments, the computer-generated model of an implant can include data formatted to be provided to a computer numerical control (“CNC”) system, a three-dimensional printer, or any other suitable system that is configured to machine or otherwise construct a designed implant.

In some instances, contralateral image information or images from a database of ideal anatomy can be used to supplement, or otherwise decrease, the need for interpolation near metal artifacts. Furthermore, the contralateral information may be used as a guide to restore the normal anatomy. Decreasing the need for interpolation would decrease the time needed for engineers to design the 3D models, which would improve the accuracy of instrumentation based on these models in addition to allowing more accurate manufacture of custom implants. This accurate anatomic information, acquired across a breadth of patients, has the potential to facilitate the design of implants that would be available off-the-shelf and not customized.

In still other aspects, the generated report can provide information about the subject's bone architecture, generally, as indicated at step 412. For instance, the report can include information, such as images, data, or information derived therefrom, that indicates a bone architecture of the subject, such as a bone density or a bone volume, as well as information about other tissues. Such a report can be advantageous for patients undergoing revision surgery, whereby information about the remaining bone or bone quality can be utilized to accurately plan for and execute a revision surgery. As another example, the report can include a computer-generated model of the subject's bone, surrounding anatomy, or both.

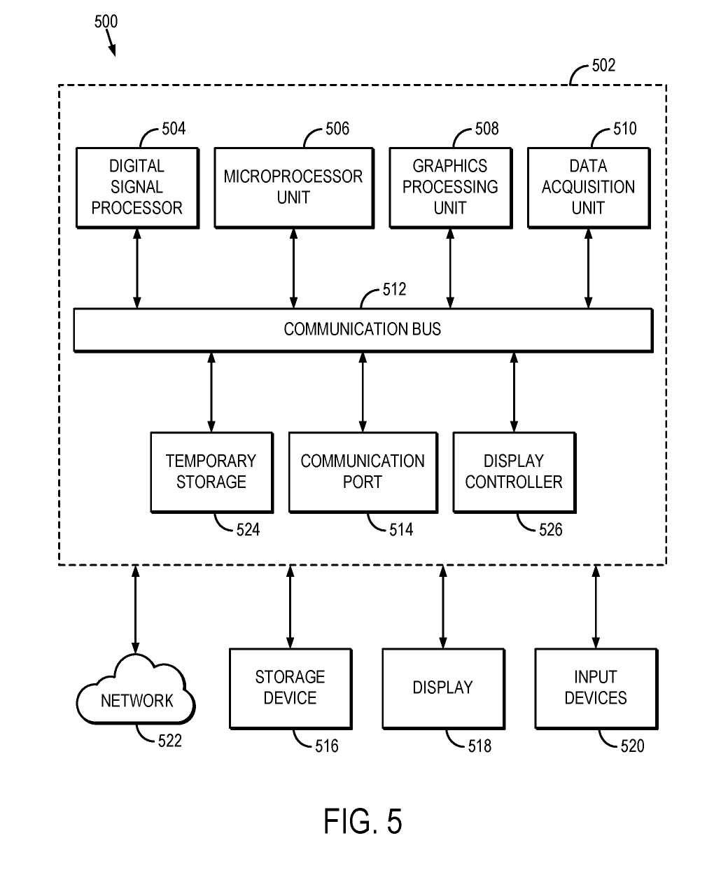

Referring now to FIG. 5, a block diagram of an example computer system 500 that can be configured to generate reports in accordance with the methods described above, is illustrated. The image data to be processed can be provided to the computer system 500 from the respective medical imaging systems, such as an x-ray imaging system or an MRI system, or from a data storage device, and are received in a processing unit 502.

In some embodiments, the processing unit 502 can include one or more processors. As an example, the processing unit 502 may include one or more of a digital signal processor (“DSP”) 504, a microprocessor unit (“MPU”) 506, and a graphics processing unit (“GPU”) 508. The processing unit 502 can also include a data acquisition unit 510 that is configured to electronically receive image data to be processed, which may include images, k-space data, or x-ray attenuation data. The DSP 504, MPU 506, GPU 508, and data acquisition unit 510 are all coupled to a communication bus 512. As an example, the communication bus 512 can be a group of wires, or a hardwire used for switching data between the peripherals or between any component in the processing unit 502.

The DSP 504 can be configured to receive and processes the image data. The MPU 506 and GPU 508 can also be configured to process the image data in conjunction with the DSP 504. As an example, the MPU 506 can be configured to control the operation of components in the processing unit 502 and can include instructions to perform processing of the image data on the DSP 504. Also as an example, the GPU 508 can process image graphics.

In some embodiments, the DSP 504 can be configured to process the image data received by the processing unit 502 in accordance with the methods described above. Thus, the DSP 504 can be configured to identify objects in the image data, to remove the objects from the image data, and to generate reports based on the processed image data. The DSP 504 can also be configured to generate image fusion data by fusing together, or otherwise combining, image data acquired with different imaging modalities or from the same imaging modality, but processed differently. Likewise, the DSP 504 can also be configured to identify objects in the image fusion data, to remove the objects from the image fusion data, and to generate reports based on the processed or unprocessed image fusion data

The processing unit 502 preferably includes a communication port 514 in electronic communication with other devices, which may include a storage device 516, a display 518, and one or more input devices 520. Examples of an input device 520 include, but are not limited to, a keyboard, a mouse, and a touch screen through which a user can provide an input.

The storage device 516 is configured to store image data, whether provided to or processed by the processing unit 502. The display 518 is used to display image data, such as images that may be stored in the storage device 516, and other information. Thus, in some embodiments, the storage device 516 and the display 518 can be used for displaying the image data before and after processing and for outputting other information, such as data plots or other reports generated based on the methods described above.

The processing unit 502 can also be in electronic communication with a network 522 to transmit and receive image data, generated reports, and other information. The communication port 514 can also be coupled to the processing unit 502 through a switched central resource, for example the communication bus 512.

The processing unit 502 can also include a temporary storage 524 and a display controller 526. As an example, the temporary storage 524 can store temporary information. For instance, the temporary storage 524 can be a random access memory.

The present invention has been described in terms of one or more preferred embodiments, and it should be appreciated that many equivalents, alternatives, variations, and modifications, aside from those expressly stated, are possible and within the scope of the invention.

1. A method for generating a report that provides information about a revision surgery plan or guide based on image data acquired with a medical imaging system, the method comprising:
(a) providing to a computer system, image data of a subject acquired with a medical imaging system;
(b) processing the provided image data with the computer system to identify at least one object implanted in the subject's anatomy;
(c) processing the provided image data with the computer system to remove the identified at least one object from the image data; and
(d) generating a report with the computer system based on the processed image data, wherein the report provides information about a revision surgery plan specific to the subject.

2. The method as recited in claim 1, wherein the medical imaging system is an x-ray imaging system and the image data comprises one of images reconstructed from data acquired with the x-ray imaging system or x-ray attenuation data acquired with the x-ray imaging system.

3. The method as recited in claim 1, wherein the medical imaging system is a magnetic resonance imaging (MRI) system and the image data comprises one of images reconstructed from data acquired with the MRI system or k-space data acquired with the MRI system.

4. The method as recited in claim 1, wherein providing the image data to the computer system comprises one of retrieving previously acquired image data from a data storage device or acquiring the image data with the medical imaging system.

5. The method as recited in claim 4, wherein acquiring the image data with the medical imaging system comprises acquiring the image data using a data acquisition that is optimized to reduce image artifacts.

6. The method as recited in claim 1, wherein the at least one object includes an implant composed of a material that significantly attenuates x-rays.

7. The method as recited in claim 6, wherein the material includes at least one of a metal, a metal alloy, a ceramic, or a plastic.

8. The method as recited in claim 1, wherein the report generated in step (d) includes a computer-generated model of at least one bone in the subject, the computer-generated model being computed based on the processed image data.

9. The method as recited in claim 1, wherein the report generated in step (d) includes at least one of a patient specific instrumentation or guide.

10. A method for generating a report that provides information for designing an implant for use in a revision surgery based on image data acquired with a medical imaging system, the method comprising:
(a) providing to a computer system, image data of a subject acquired with a medical imaging system;
(b) processing the provided image data with the computer system to identify at least one object implanted in the subject's anatomy;
(c) processing the provided image data with the computer system to remove the identified at least one object from the image data; and
(d) generating a report with the computer system based on the processed image data, wherein the report provides information for designing a subject-specific implant for use in a revision surgery.

11. The method as recited in claim 10, wherein medical imaging system is an x-ray imaging system and the image data comprises one of images reconstructed from data acquired with the x-ray imaging system or x-ray attenuation data acquired with the x-ray imaging system.

12. The method as recited in claim 10, wherein the medical imaging system is a magnetic resonance imaging (MRI) system and the image data comprises one of images reconstructed from data acquired with the MRI system or k-space data acquired with the MRI system.

13. The method as recited in claim 10, wherein providing the image data to the computer system comprises one of retrieving previously acquired image data from a data storage device or acquiring the image data with the medical imaging system.

14. The method as recited in claim 13, wherein acquiring the image data with the medical imaging system comprises acquiring the image data using a data acquisition that is optimized to reduce image artifacts.

15. The method as recited in claim 10, wherein the at least one object includes an implant composed of a material that significantly attenuates x-rays.

16. The method as recited in claim 15, wherein the material includes at least one of a metal, a metal alloy, a ceramic, or a plastic.

17. The method as recited in claim 10, wherein the report generated in step (d) includes a computer-generated model of the subject-specific implant, the computer-generated model being computed based on the processed image data.

18. A method for generating a report that provides information about a subject's bone architecture based on image data acquired with a medical imaging system, the method comprising:
(a) providing to a computer system, image data of a subject acquired with a medical imaging system;
(b) processing the provided image data with the computer system to identify at least one object implanted in the subject's anatomy;
(c) processing the provided image data with the computer system to remove the identified at least one object from the image data; and
(d) generating a report with the computer system based on the processed image data, wherein the report provides information about the subject's bone architecture.

19. The method as recited in claim 18, wherein the medical imaging system is an x-ray imaging system and the image data comprises one of images reconstructed from data acquired with the x-ray imaging system or x-ray attenuation data acquired with the x-ray imaging system.

20. The method as recited in claim 18, wherein the medical imaging system is a magnetic resonance imaging (MRI) system and the image data comprises one of images reconstructed from data acquired with the MRI system or k-space data acquired with the MRI system.

21. The method as recited in claim 18, wherein providing the image data to the computer system comprises one of retrieving previously acquired image data from a data storage device or acquiring the image data with the medical imaging system.

22. The method as recited in claim 21, wherein acquiring the image data with the medical imaging system comprises acquiring the image data using a data acquisition that is optimized to reduce image artifacts.

23. The method as recited in claim 18, wherein the at least one object includes an implant composed of a material that significantly attenuates x-rays.

24. The method as recited in claim 23, wherein the material includes at least one of a metal, a metal alloy, a ceramic, or a plastic.

25. The method as recited in claim 18, wherein the provided image data comprises dual-energy image data.

26. The method as recited in claim 18, wherein step (d) includes performing a material decomposition on the processed image data with the computer system to identify a bone tissue of the subject.

27. The method as recited in claim 26, wherein the generated report includes at least one of a bone density and a bone volume of a subject.

28. A method for generating a report that provides information about a revision surgery plan, revision surgery guide, subject-specific implant for use in a revision surgery, or the subject's bone architecture based on image data acquired with a medical imaging system, the method comprising:
(a) providing to a computer system, image data of a subject acquired with at least one medical imaging system;
(b) generating image fusion data by combining the image data with the computer system, whereby the image fusion data enhances a depiction of at least one object implanted in the subject's anatomy relative to the image data; and
(c) generating a report with the computer system based on the image fusion data, wherein the report provides information about at least one of a revision surgery plan specific to the subject, designing a subject-specific implant for use in a revision surgery, or the subject's bone architecture.

29. The method as recited in claim 28, wherein the at least one medical imaging system includes at least one of an x-ray imaging system, a magnetic resonance imaging (MRI) system, or an ultrasound imaging system.

30. The method as recited in claim 29, wherein the image data comprises at least one of images reconstructed from data acquired with the x-ray imaging system, x-ray attenuation data acquired with the x-ray imaging system, images reconstructed from data acquired with the MRI system, k-space data acquired with the MRI system, or images acquired with the ultrasound imaging system.

31. The method as recited in claim 28, wherein the image data comprises image data associated with at least two different imaging modalities.

32. The method as recited in claim 28, wherein the image data comprises image data associated with a single imaging modality that have been differently processed.
